(19) United States
(12) Patent Application Publication
(10) Pub. No.: US 2011/0097316 A1
(43) Pub. Date: Apr. 28, 2011

(54) COMPOSITION TO BE ABSORBED THROUGH MUCOUS TISSUE

(76) Inventor: Peter Hernuss, Wien (AT)

(21) Appl. No.: 12/735,703

(22) PCT Filed: Feb. 13, 2009
(86) PCT No.: PCT/EP2009/051682

§ 371 Date: Dec. 14, 2010

(30) Foreign Application Priority Data
Feb. 13, 2008 (AT) A 238/2008

(51) Int. Cl. A61K 38/48; A61P 43/00

(52) U.S. Cl. 424/94.65; 424/94.63

(57) ABSTRACT

The invention relates to a composition to be absorbed via mucous tissue, containing at least one hormone cleavable by proteinases and at least one proteinase or mixture of proteinases, where the at least one hormone is kept separate from the proteinase or mixture of proteinases in the composition, and furthermore to the use of the composition and to a kit for administering the composition.

The invention relates to a composition to be absorbed through mucous tissue, which composition supplies substances for the stimulation of hormone production in the human body in addition to enhancing general well-being, and further to the use of said composition in preparing a nutritional supplement and to a kit for administering the composition.

Nutritional supplements are becoming increasingly popular. It is their aim to support a healthy way of life or to satisfy an increased need for certain nutrients due to a demanding way of life or to nutritional deficits. Partially they also have an “anti-aging” effect, and there is increasing demand for such anti-aging products, which slow down the aging process and provide better quality of life for the aged.

It is for instance known that the growth hormone level decreases with age. If the synthesis of growth hormone is assisted from the age of about forty onwards, a significant improvement of fitness and physiological processes has been found.

Somatotropin (hGH, hypophyseal or human growth hormone) is a hormone which is formed in the front lobe of the pituitary gland and regulates the growth process of children and the course of numerous bodily functions in the adult. A somatotropin deficit in youth causes stunted growth, overproduction causes acromegaly (gigantism; increased growth of the acra, i.e. hands and feet). The human growth hormone is a single polypeptide with a molecular weight of 21,500 Dalton consisting of 191 amino acids of known sequence with a number of disulfide bonds.

If hGH is administered orally it is immediately destroyed in the stomach and thus has no effect on the human body. Intake of the complete growth hormone molecule via the mucous membranes is not possible since the molecule cannot pass the membranes due to its size. Somatotropin is therefore usually administered by injection. Similar problems occur with other peptide hormones whose size prohibits intake via the mucous membranes and which are destroyed by gastric acid and thus are ineffective if taken orally. Shorter polypeptides can be supplied to the organism via the mucous membranes or the skin, however. This form of intake is limited by diverse factors, above all by the size of the peptide molecule. For polypeptides a molecular size corresponding to a length of roughly 80 amino acids and a molecular weight of roughly 10,000 Dalton presents the upper limit for intake via the mucous membrane.

It is therefore the object of the present invention to supply a composition which stimulates hormone production and causes an improvement of general condition.

This object is achieved by the invention by providing a composition as initially mentioned, which contains at least one hormone that is cleavable by proteinases and at least one proteinase or a mixture of proteinases, the at least one hormone being provided in the composition separately from the proteinase or proteinase mixture. Proteinases are enzymes which catalyse the hydrolytic cleavage of the peptide bond in the interior of proteins or peptides. In WO 2004/100981 A1 a composition is described, which contains one or more hydrolases and one or more antioxidants.

The polypeptide chain of the hormone is initially split into larger, then into smaller segments (cleavage products) by the proteinase or proteinase mixture, thus permitting intake of biologically or biochemically effective protein segments of long polypeptide chains through mucous tissue such as the nasal mucous membrane or the mucosa of the mouth. The hormones which are relevant for the invention are primarily peptide hormones or glycoprotein hormones whose molecular size prior to enzymatic cleavage prohibits their intake via mucous membranes.

Besides polypeptide segments effective in the general improvement of well-being, other cleavage products are provided for stimulation of hormone production in the body, which due to their size (smallness) can be absorbed via the mucous membrane.

When the proteinase acts on the hormone, which usually consists of a long polypeptide chain, the hormone is split into cleavage peptides of lesser molecular weight, that is within a period of less than one minute into peptide fragments of mass between 0.6 to 6 kD (corresponding to 5 to 50 amino acids), which then can be taken in by the body via the mucous tissue. These cleavage peptides or hormone fragments stimulate the body or the respective gland, to synthesize the complete hormone originally contained in the composition of the invention. In several studies (e.g. a paper by Dr. Hans-Günter Kugler in Co'Med August 2002) it was found, for instance, that an addition of L-glutamine increased the hGH level, improved protein synthesis and significantly strengthened the immune system. It is also known that L-arginine enhances hGH production by inhibiting the secretion of somatostatin (a growth hormone inhibitor) and by stimulating the pituitary gland, which secretes human growth hormone, to produce hGH. Thus peptide fragments obtained by cleavage of hGH are capable of stimulating hormone production. This has also been found in the case of peptide hormones other than somatotropin. The mechanism of action of the composition proposed by the invention may be compared for instance to that of a vaccination, where fragments of viruses and/or antibodies are administered to the body in order to stimulate the production of antibodies.

During storage of the composition of the invention the hormone and the proteinase or proteinase mixture must be kept separate to avoid premature hydrolysis of the hormone.

The at least one hormone is preferably selected from the group containing the growth hormone somatotropin (hGH), erythro-poietin (EPO) and human chorionic gonadotropin (hCG). Enhanced synthesis of somatotropin improves memory performance and well-being. Erythropoietin is a hormone responsible for the forming of erythrocytes and is used in the therapy of anaemia, especially in cancer therapies. It is furthermore used to improve physical strength and endurance, abusively so in sports for the doping of top athletes. Chorionic gonadotropin is a hormone which is produced by the body during pregnancy and is responsible for the embedding of the embryo in the placenta. Enhancement of hCG synthesis can reduce the risk of miscarriage in the first weeks of pregnancy and improve fertility.

The mechanism of action of the composition proposed by the invention will now be explained using somatotropin as an example. The biologically active effects of the somatropic hormone are not caused or achieved by the molecule in its entirety, but by certain sites which can dock directly onto DNA. There these so-called DNA response elements act on the transcription process. Only certain regions of the molecule of the growth hormone are involved in this while other areas are responsible for the stability of the molecule, etc. From this it may be inferred that the same kind of mechanism is present in other hormones, where one or more fragments of the hormone cause the stimulative effect. If the hormone is split into small fragments, which can be absorbed via mucous membranes, the same effect will be obtained, since those parts of the hormone causing the corresponding effects on DNA will be provided. Thus the structure of the hormone is split, but the effect will be unimpaired since fragments remain which contain regions that cause the desired effect. It could even be the case that fragmentation of the hormone molecule will enhance the effect, as the smaller fragments are less impeded by steric or space constraints and may dock onto the receptors faster and more easily, resulting in enhanced stimulation of hormone production. It is for instance known that coagulation factors (e.g. factor VII, factor VIII) are only activated by proteolytic cleavage and that their fragments will cause the desired effect.

In a preferred variant of the invention the proteinase or mixture of proteinases is selected from the group containing papain, bromelain, ficin and asclepain.

Papain is a proteinase which is derived from the latex of unripe papayas, a crystalline polypeptide with a molecular weight of 23,350 Da consisting of a chain of 212 amino acid remnants with four disulfide bonds whose sequence and spatial structure are known. Other vegetable enzymes similar to papain are bromelain (from pineapple), ficin (from figs) and asclepain (from asclepia speciosa of the milkweed family). These proteinases are obtainable in a simple manner and are well-tolerated in general.

A particularly preferred intake of the composition is via the mucous membranes of the mouth. In this case the at least one hormone and the at least one proteinase or proteinase mixture come into contact with each other only in the oral cavity. The environment prevalent in the mouth will ensure rapid cleavage of the hormone within a few seconds. The products of cleavage are absorbed via the mucous membrane of the mouth, while proteinase and remaining unsplit hormone cannot penetrate the mucous membrane due to their molecular size, and are ingested and decomposed in the stomach without generating noxious by-products.

It has been found that the hormone is split most quickly if the at least one hormone and the at least one proteinase or proteinase mixture are present in equimolar ratio.

In addition, the composition of the invention preferably contains pharmaceutically well-tolerated auxiliary substances, in particular solvents, preservation agents and flavouring agents, which will improve storability and taste of the composition and permit easier administering.

In a preferred variant of the invention the composition contains additional vitamins and/or trace elements, which are beneficial to the health and well-being of the user.

According to the invention, the composition of the invention, which contains a hormone and at least one proteinase or proteinase mixture, is used as a nutritional supplement for the improvement of mental and physical well-being. It has been found that a combination of somatotropin with at least one proteinase, in particular papain, or a mixture of two or more proteinases, will counteract aging processes.

For simple administering of the composition of the invention via the mucous membrane of the mouth a kit is provided, which contains a first spray can with at least one hormone and a second spray can with at least one proteinase or proteinase mixture. The user simply sprays the solution containing one or more hormones from the first spray can and immediately thereafter the solution containing the proteinase from the second spray can onto the buccal mucous membrane. Neither the hormone nor the proteinase can reach the organism via the mucous membrane, while the cleavage products produced by the hydrolytic cleavage of the hormone by means of the proteinase or proteinase mixture are absorbed due to their smaller molecular size and will stimulate hormone production.

The invention will now be further described using an example which is not to be interpreted restrictively.

The nutritional supplement according to the invention contains the human growth hormone somatotropin (hGH) besides other compounds such as preservation and flavouring agents. It is filled into a first spray can as a solution. A second spray can is filled with a sprayable solution containing the proteinase papain.

The growth hormone is first sprayed onto the buccal mucous membrane from the first spray can. Subsequently the proteinase from the second spray can is sprayed onto the same area of the mucous membrane.

The following table 1 shows the composition of one spraying act (i.e. approximately 0.1 ml fluid) from each spray can.

TABLE 1

| Spray 1 | | | Spray 2 | | |
|---|---|---|---|---|---|
| Content of one spray stroke | | | Content of one spray stroke | | |
| Somatotropin (hGH) | 300 | ng | Papain | 300 | ng |
| Aqua purificata | 0.09 | ml | Aqua purificata | 0.075 | ml |
| Sodium benzoate | 0.015 | mg | Sodium benzoate | 0.015 | mg |
| Potassium sorbate | 0.025 | mg | Potassium sorbate | 0.025 | mg |
| Xylitol | 15 | mg | Passiflora Codi | 2.5 | mg |
| | | | Xanthan FNCS 1165/01, 04 | 0.2 | mg |
| | | | Xylitol | 15 | mg |
| | | | 96% Ethanol | 10.5 | mg |

If somatotropin is taken orally in a conventional way it is destroyed by gastric juices and can therefore not produce any of the desired effects in the body. The somatotropin molecule with a mean molecular weight of 21,500 Da is too large to be absorbed via human or animal mucous membranes.

The growth hormone is therefore first applied to the mucous membrane, preferably the mucous membrane of the mouth, and immediately afterwards the proteinase, i.e. papain, is sprayed on. The proteinase now has the task of enzymatically splitting the hGH into individual protein fragments, which can be taken in via the mucous membrane. Protein fragments of roughly 80 amino acids are immediately absorbed by the mucous membrane due to their size and do not undergo further hydrolytic splitting, since papain on account of its molecular size is not absorbed via the mucous membrane of the mouth.

Cleavage of the growth hormone under the action of papain has been investigated in a series of tests. The molecular masses of the cleavage peptides produced by the action of papain over diverse periods of time were measured by means of mass-spectrometry.

The tests were carried out for three different hGH/papain mixing ratios. Besides the equimolar ratio (hGH:papain=1:1) two sub-equimolar ratios (hGH:papain=4:1 and 10:1) were investigated. A hGH-sample without added papain was used as a reference.

For the time-series tests by means of MALDI-TOF mass spectrometry 10 μL of hGH stock solution (with a concentration of 4 mg/mL) were mixed with 180 μL 20 mM ammonacetate solution in a reaction vessel of 500 μL volume. The reaction was started by adding 10 μL of the papain stock solution (with a concentration of 4 mg/mL). Papain as well as hGH thus were present at a concentration of 0.2 mg/mL. This corresponds approximately to an equimolar mixing ratio of hGH and papain.

Enzymatic peptidase reaction of papain was stopped after different time intervals. The intervals used were 15, 30, 60, 120, 180, and 300 seconds. The stopping of the enzyme action and the cleavage reaction was performed in two different ways in parallel experiments.

(1) Spotting the hGH/papain solution onto the MALDI-target and immediately adding trifluoroacetic acid (TFA). Here stopping of the enzyme reaction was expected on account of low pH.

(2) Adding phenylmethylsulphonylfluoride (PMSF) as enzyme inhibitor to the hGH/papain solution at a selected point in time.

Altogether six independent test series were carried out for determining the development over time of the enzymatic cleavage process, corresponding to the three hGH/papain mixing ratios times the two types of enzyme inhibition described above.

Determination of the reaction products was carried out by measuring the molecular mass of the cleavage peptides by means of MALDI-TOF-MS. Since papain has no specific splitting site peptide sequences could not be determined from the peptide masses.

The following results were obtained from the mass-spectrometric investigation of the cleavage peptides of the six test series.

In aqueous solutions under neutral pH conditions papain will split hGH into peptides within a time period of less than one minute to a large extent though presumably not completely. After roughly 3 minutes there is no longer any of the original hGH left. Due to the unspecific splitting of hGH a number of fragments are produced, which are not appreciably split further within a time period of 7 minutes. Their molecular masses lie in a range between 600 and 4,500 Da, corresponding to peptide sizes of 5 to 30 amino acids. These patterns are reproducible and are not substantially changed by a change in the amount of enzyme added (1:1 or 1:4 or 1:10).

Protein fragments which are administered to the body in this way support the production of growth hormone in the pituitary gland. Growth hormones are essential for well-being, in particular for slowing down aging processes and enhancing mental fitness.

It has been found that regular application of the composition according to the invention will improve memory performance, hair growth, will delay the greying of hair and will in general result in increased mental and physical well-being. In a first test series with 101 subjects, of which 40 were given a placebo and thus formed a control group, the test group proper consisting of 31 men and 30 women between 25 and 35 years of age, it was found that an intake of the composition of the invention over an average period of 9 months resulted in a significant increase of energy and endurance, of everyday productivity, of the sexual functions and of the growth of hair and nails, as compared with the control group. Gender-dependence of the effect has not been found.

It should be understood that the example described above is not meant to be restrictive. The composition of the invention may for instance vary; instead of papain another protein-cleaving enzyme, such as bromelain, ficin or asclepain or mixtures of diverse proteinases, may for instance be used. Also mixtures of hormones, for instance EPO and hGH, are possible. Neither will application be restricted to the mucous membranes of the mouth; the intake of cleavage peptides could also occur via the mucous membranes of the nose. Instead of sprays other forms of administration are conceivable. Hormone and proteinase could be presented in the form of a lozenge, with the hormone or the hormones being separated from the proteinase by a water-soluble barrier, for instance by encapsulation, and being released and reacting when the lozenge dissolves in the mouth, the cleavage products again being absorbed via the mucous membrane of the mouth. The composition could also be administered in capsules which release their content in the intestine.

1. A composition for absorption through mucous tissue comprising at least one hormone which can be split by proteinases and at least one proteinase or mixture of proteinases, where the at least one hormone is kept separate from the proteinase or mixture of proteinases in the composition.

2. The composition according to claim 1, wherein the at least one hormone is selected from the group consisting of the growth hormone somatotropin (hGH), erythropoietin (EPO) and human chorionic gonadotropin (hCG).

3. The composition according to claim 1, wherein the proteinase or mixture of proteinases is selected from the group consisting of papain, bromelain, ficin and asclepain.

4. The composition according to claim 1, wherein at least one hormone and the at least one proteinase are present in equimolar ratio.

5. The composition according to claim 1, including pharmaceutically well-tolerated auxiliary substances, in particular solvents, preservation agents and flavouring agents.

6. The composition according to claim 1, including vitamins and/or trace elements.

7. Use of a composition according to claim 1 for the production of a nutritional supplement for improving physical and mental well-being, the composition preferably containing somatotropin.

8. Kit for administering a composition according to claim 1 via the mucous membrane of the mouth, comprising a first spray can containing at least one hormone and a second spray can containing at least one proteinase or mixture of proteinases.
